(19) United States
(12) Patent Application Publication
(10) Pub. No.: US 2024/0225832 A9
(43) Pub. Date: Jul. 11, 2024

(54) Apical Pad for Prosthetic Heart Valve

(71) Applicant: Tendyne Holdings, Inc., St. Paul, MN (US)

(72) Inventor: Amy Marie Danielson, St. Paul, MN (US)

(73) Assignee: Tendyne Holdings, Inc., St. Paul, MN (US)

(21) Appl. No.: 18/486,131

(22) Filed: Oct. 12, 2023

Related U.S. Application Data
(60) Provisional application No. 63/380,841, filed on Oct. 25, 2022.

(51) Int. Cl. A61F 2/24

(52) U.S. Cl. CPC A61F 2/243; A61F 2220/0016

(57) ABSTRACT

An epicardial anchor device may include a base defining a radial slot, and a top component including a first piece and a second piece each operably coupled to the base. The first piece may include a first recess and the second piece may include a second recess. The epicardial anchor device may have (i) an unpinned condition in which the first piece and the second piece are spaced from each other a first distance so that a tether may be laterally slid within the radial slot and between the first piece and the second piece, and (ii) a pinned condition in which the first piece and the second piece are spaced from each other a second distance so that the tether may be confined within an aperture defined by the first recess and the second recess, the second distance being smaller than the first distance.

### CROSS-REFERENCE TO RELATED APPLICATIONS

This application claims benefit to the priority of U.S. Provisional Patent Application No. 63/380,841, filed Oct. 25, 2022, the disclosure of which is hereby incorporated by reference herein.

### BACKGROUND OF THE DISCLOSURE

Embodiments are described herein that relate to devices and methods for anchoring a medical device such as a prosthetic heart valve replacement.

Some known devices for anchoring a medical device, such as, for example, a prosthetic heart valve (e.g., mitral valve) can include securing one or more tethers extending from the medical device to body tissue. For example, one or more tethers can extend from a prosthetic heart valve through an opening in the ventricular wall of the heart. Some known methods of anchoring or securing the tethers can include the use of staples or other fasteners that engage or pierce tissue near the puncture site. Such devices can have relatively large profiles and be difficult to easily deliver percutaneously to the desired anchoring site. Some known methods of securing a prosthetic heart valve can include suturing the tethers extending from the valve to body tissue or tying the suture ends. Such devices and methods can be difficult to maneuver to secure the tether(s) with the desired tension,

Other known devices may include anchors that include a central aperture through which the tether is passed, with the tether being secured within the central aperture after being passed therethrough. In some circumstances, it may be desirable to disconnect the tether from the anchor in order to re-tension the tether, or in order to replace the original anchor with another anchor. In these circumstances, it may be desirable to have an anchor that facilitates easy and rapid removal of the anchor from the tether and/or securement of the anchor to the tether. Some known devices may include relatively large, static structures that require correspondingly large incisions in the patient to deliver. In some circumstances, it may be desirable to have an anchor that may be delivered in a relatively small condition and expanded or actuated when the anchor is at or adjacent to the heart.

### BRIEF SUMMARY OF THE DISCLOSURE

According to one aspect of the disclosure, an epicardial anchor device includes a base and a top component. The base may define a radial slot. The top component may include a first piece and a second piece each operably coupled to the base, the first piece including a first recess and the second piece including a second recess, the first and second recesses facing each other in an assembled condition of the epicardial anchor device. The epicardial anchor device may have (i) an unpinned condition in which the first piece and the second piece are spaced from each other a first distance so that a tether may be laterally slid within the radial slot and between the first piece and the second piece, and (ii) a pinned condition in which the first piece and the second piece are spaced from each other a second distance so that the tether may be confined within an aperture defined by the first recess and the second recess, the second distance being smaller than the first distance. A pin may be coupled to the first piece and may extend into the first recess. In the pinned condition, the pin may traverse the aperture defined by the first recess and the second recess, a free end of the pin being received within a pin channel defined by the second piece, the pin channel being adjacent to the second recess. A rail may have a first end and a second end, wherein in the assembled condition of the epicardial anchor device, the first end of the rail is received within a first rail channel defined by the first piece, and the second end of the rail is received within a second rail channel defined by the second piece. In the absence of applied force, engagement of the rail with the first piece and with the second piece may prevent the epicardial anchor device from transitioning between the pinned condition and the unpinned condition. The rail may include a plurality of troughs and peaks, and each of the first rail channel and the second rail channel may include a plurality of troughs and peaks, the peaks of the rail being sized and shaped to be received within the troughs of the first rail channel and the troughs of the second rail channel. The peaks of the rail may be deformable so that, upon application of applied force to the first piece and/or the second piece, the peaks of the rail displace from the corresponding troughs of the first rail channel and the second rail channel to adjacent troughs of the first rail channel and the second rail channel. The base may include a first slot on a first side of the radial slot and a second slot on a second side of the radial slot opposite the first side, the first piece having a first protrusion configured to be received within the first slot, and the second piece having a second protrusion configured to be received within the second slot. The first slot and the second slot may each have a narrow portion and a wide portion, and the first protrusion and the second protrusion may each have a narrow portion and a wide portion, the wide portion of the first protrusion sized to be received through the wide portion of the first slot, and the wide portion of the second protrusion sized to be received through the wide portion of the second slot. The wide portion of the first protrusion may be larger than the narrow portion of the first slot, and the wide portion of the second protrusion may be larger than the narrow portion of the second slot.

According to another aspect of the disclosure, an epicardial anchor device may include a base component and a plurality of arms. The base component may have a base with a bottom surface configured to contact a heart of a patient, the base defining an outer perimeter, the base component defining a tether-receiving passageway configured to receive a tether therethrough. The plurality of arms may be hingedly coupled to the base, the plurality of arms being rotatable relative to the base between (i) a stowed condition in which none of the plurality of arms extend radially outward beyond the outer perimeter of the base and (ii) a deployed condition in which each of the plurality of arms extend radially outward beyond the outer perimeter of the base. Each of the plurality of arms may have a main body portion and a free end portion angled relative to the main body portion. In the deployed condition of the plurality of arms, the free end portion of each of the plurality of arms may be positioned to contact the heart of the patient while the bottom surface of the base of the base component simultaneously contacts the heart of the patient. A plurality of hinge posts may extend from the base of the base component in an upward direction away from the bottom surface of the base of the base component, each of the plurality of arms being hingedly coupled to the base at corresponding ones of the hinge posts. Each of the plurality of hinge posts may include two protrusions, and each of the plurality of arms may include two loops configured to receive the two protrusions of a corresponding one of the plurality of hinge posts. The base component may include a center post extending in an upward direction away from the bottom surface of the base of the base component, the tether-receiving passageway extending through the center post. A cover may receive the center post therethrough, the cover having a plurality of extensions extending radially outward from the center post, a gap being defined between each circumferentially adjacent pair of extensions. In an unlocked condition of the cover and in the stowed condition of the plurality of arms, each arm may be positioned within a corresponding gap of the cover. The cover may be rotatable relative to the center post from the unlocked condition to a locked condition, wherein in the locked condition of the cover and in the deployed condition of the plurality of arms, each extension overlies a corresponding one of the plurality of arms so that each of the plurality of arms is prevented from transitioning from the deployed condition to the stowed condition. Each of the extensions may include a lip extending downwardly toward the bottom surface of the base of the base component, and in the locked condition of the cover and in the deployed condition of the plurality of arms, each lip may contact a corresponding one of the plurality of arms.

According to another aspect of the disclosure, an epicardial anchor device includes a base defining (i) a radial tether slot extending through a top and bottom surface of the base and (ii) two arm slots extending through the top surface of the base. The anchor device may include wo extension arms that each have an arcuate contact arm, a post, and a beam extending between the contact arm and the post. Each post may be sized and shaped to be received within and slide along a corresponding one of the two arm slots. A cap may be mounted to the base so that the two extension arms are positioned at least partially between the cap and the base. The anchor device may have a contracted position in which the posts of the extension arms are relatively close to a longitudinal center of the base and the contact arms are aligned with an outer perimeter of the base. The anchor device may also have an extended position in which the posts of the extension arms are relatively far from the longitudinal center of the bae and the contact arms extend beyond the outer perimeter of the base. In the extended position, a bottom surface of each of the extension arms may be substantially coplanar with the bottom surface of the base. The beam of each of the two extension arms may have at least one groove, and a biasing mechanism may be positioned within the base. The biasing mechanism may interact with the at least one groove to maintain the anchor device in the contracted position in the absence of applied forces. Each of the two arm slots may include an opening that opens to the bottom surface of the base. The post of each of the two extension arms may be received within a corresponding one of the openings when the anchor device is in the extended position.

### BRIEF DESCRIPTION OF THE DRAWINGS

FIG. 1 is a cross-sectional illustration of portion of a heart with a prosthetic mitral valve implanted therein and an epicardial anchor device anchoring the mitral valve in position.

FIG. 2 is a schematic illustration of an epicardial anchor device, according to an embodiment.

FIG. 3A is a top perspective view of an epicardial anchor device, according to another embodiment.

FIG. 3B is a top view of the epicardial anchor device of FIG. 3A.

FIG. 3C is an exploded view of the epicardial anchor device of FIG. 3A.

FIG. 3D is a cross-sectional perspective view of the epicardial anchor device of FIG. 3A with a locking pin of the device shown in a first position.

FIG. 3E is a cross-sectional side view of the epicardial anchor device of FIG. 3A with the locking pin of the device shown in the first position.

FIG. 3F is a cross-sectional bottom perspective view of the epicardial anchor device of FIG. 3A with the locking pin shown in a second position.

FIGS. 3G and 3H are a top perspective and a bottom perspective view, respectively, of a hub member of the epicardial anchor device of FIG. 3A.

FIG. 3I is an enlarged top view of a portion of the pericardial pad device of FIG. 3A.

FIG. 4 is a perspective view of the epicardial anchor device of FIG. 3A with a delivery device coupled thereto.

FIGS. 5A-B are top and perspective views, respectively, of an anchor device according to an embodiment of the disclosure.

FIGS. 5C-D are top and perspective views, respectively, of a base component of the anchor device of FIGS. 5A-B.

FIGS. 5E-G are top, side, and perspective views, respectively, of a top component of the anchor device of FIGS. 5A-B.

FIGS. 5 H-I are top and perspective views, respectively, of a rail component of the anchor device of FIGS. 5A-B.

FIG. 6A is a perspective view of an anchor device according to another embodiment of the disclosure.

FIGS. 6B-D are top, side, and perspective views, respectively, of a base component of the anchor device of FIG. 6A.

FIGS. 6E-G are perspective, top, and side views of a hinge arm of the anchor device of FIG. 6A.

FIGS. 6H-J are bottom, side, and perspective views of a top component of the anchor device of FIG. 6A.

FIGS. 7A-C are top, perspective, and side views, respectively, of an anchor device according to an embodiment of the disclosure.

FIGS. 7D-G are perspective, top, bottom, and side views, respectively, of a base of the anchor device of FIGS. 7A-C.

FIGS. 7H-L are bottom perspective, top, top perspective, end, and sides views, respectively, of an extension arm of the anchor device of FIGS. 7A-C.

FIGS. 7M-O are side, perspective, and bottom views, respectively, of a cap of the anchor device of FIGS. 7A-C.

### DETAILED DESCRIPTION OF THE DISCLOSURE

As used in this specification, the singular forms “a,” “an” and “the” include plural referents unless the context clearly dictates otherwise. Thus, for example, the term “a member” is intended to mean a single member or a combination of members, and “a material” is intended to mean one or more materials, or a combination thereof.

As used herein, the words “proximal” and “distal” refer to a direction closer to and away from, respectively, an operator of, for example, a medical device. Thus, for example, the end of the medical device closest to the patient's body (e.g., contacting the patient's body or disposed within the patient's body) would be the distal end of the medical device, while the end opposite the distal end and closest to, for example, the user (or hand of the user) of the medical device, would be the proximal end of the medical device.

In some embodiments, an epicardial pad system is described herein that can be used to anchor a compressible prosthetic heart valve replacement (e.g., a prosthetic mitral valve), which can be deployed into a closed beating heart using a transcatheter delivery system. Such an adjustable-tether and epicardial pad system can be deployed via a minimally invasive procedure such as, for example, a procedure utilizing the intercostal or subxyphoid space for valve introduction. In such a procedure, the prosthetic valve can be formed in such a manner that it can be compressed to fit within a delivery system and secondarily ejected from the delivery system into the target location, for example, the mitral or tricuspid valve annulus.

A compressible prosthetic mitral valve can have a shape, for example that features a tubular stent body that contains leaflets and an atrial cuff. This allows the valve to seat within the mitral annulus and be held by the native mitral leaflets. The use of a flexible valve attached using an apical tether can provide compliance with the motion and geometry of the heart. The geometry and motion of the heart are well-known as exhibiting a complicated biphasic left ventricular deformation with muscle thickening and a sequential twisting motion. The additional use of the apically secured ventricular tether helps maintain the prosthetic valve's annular position without allowing the valve to migrate, while providing enough tension between the cuff and the atrial trabeculations to reduce, and preferably eliminate, perivalvular leaking. The use of a compliant valve prosthesis and the special shape and features can help reduce or eliminate clotting and hemodynamic issues, including left ventricular outflow tract (LVOT) interference problems. Many known valves are not able to address problems with blood flow and aorta/aortic valve compression issues.

Structurally, the prosthetic heart valve can include: a self-expanding tubular frame having a cuff at one end (the atrial end); one or more attachment points to which one or more tethers can be attached, preferably at or near the ventricular end of the valve; and a leaflet assembly that contains the valve leaflets, which can be formed from stabilized tissue or other suitable biological or synthetic material. In one embodiment, the leaflet assembly may include a wire form where a formed wire structure is used in conjunction with stabilized tissue to create a leaflet support structure, which can have anywhere from 1, 2, 3 or 4 leaflets, or valve cusps disposed therein. In another embodiment, the leaflet assembly can be wireless and use only the stabilized tissue and stent body to provide the leaflet support structure, and which can also have anywhere from 1, 2, 3 or 4 leaflets, or valve cusps disposed therein.

The upper cuff portion may be formed by heat-forming a portion of a tubular nitinol structure (formed from, for example, braided wire or a laser-cut tube) such that the lower portion retains the tubular shape but the upper portion is opened out of the tubular shape and expanded to create a widened collar structure that may be shaped in a variety of functional regular or irregular funnel-like or collar-like shapes.

A prosthetic mitral valve can be anchored to the heart at a location external to the heart via one or more tethers coupled to an anchor device, as described herein. For example, the tether(s) can be coupled to the prosthetic mitral valve and extend out of the heart and be secured at an exterior location (e.g., the epicardial surface) with an anchor device, as described herein. An anchor device as described herein can be used with one or more such tethers in other surgical situations where such a tether may be desired to extend from an intraluminal cavity to an external anchoring site.

The prosthetic heart valve may take other forms, for example an outer stent that is coupled to an inner stent, with the inner stent housing the prosthetic leaflets, and the tether attached to a ventricular end of the inner stent. Such configurations are described in greater detail in U.S. Patent Application Publication No. 2017/0196688, the disclosure of which is hereby incorporated by reference herein. Various examples of epicardial pads and methods of using the same are described in more detail in U.S. Patent Application Publication No. 2016/0143736, the disclosure of which is hereby incorporated by reference herein.

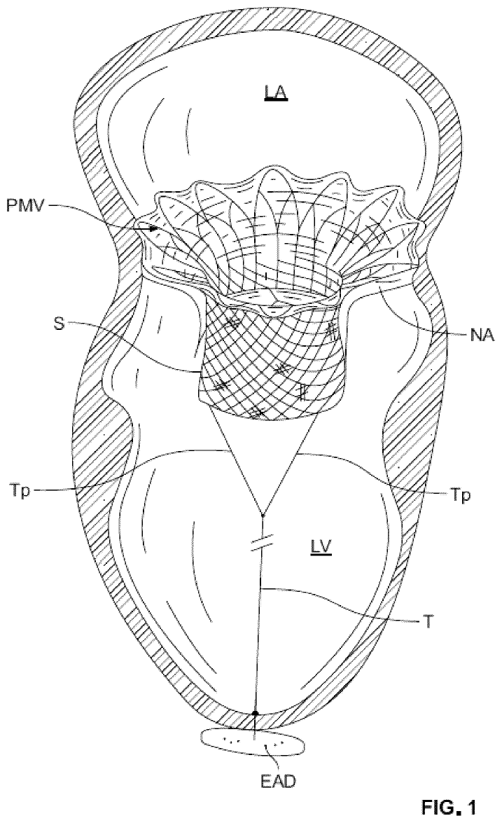

FIG. 1 is a cross-sectional illustration of the left ventricle LV and left atrium LA of a heart having a transcatheter prosthetic mitral valve PMV deployed therein, and an epicardial anchor device EAD as described herein securing the prosthetic mitral valve PMV in place. FIG. 1 illustrates the prosthetic mitral valve PMV, including a stent S, seated into the native valve annulus NA and held there using an atrial cuff of the prosthetic mitral valve PMV, the radial tension from the native leaflets, and a ventricular tether T secured with attachment portions Tp to the prosthetic mitral valve PMV and to the epicardial anchor EAD. Although FIG. 1 illustrates multiple attachment portions Tp that converge to a single ventricular tether T, in some embodiments, a single ventricular tether T may be coupled to the outflow end of the prosthetic mitral valve PMV, for example at a radially central location of the outflow end of an inner frame of the prosthetic mitral valve PMV. Various embodiments of an epicardial anchor device are described in more detail below with reference to specific embodiments.

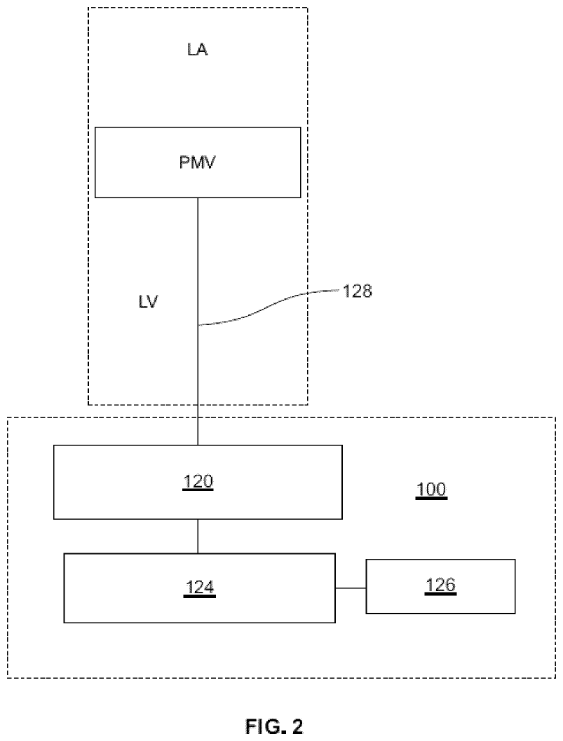

FIG. 2 is a schematic illustration of an epicardial anchor device 100 (also referred to herein as “anchor device” or “epicardial anchor”) according to an embodiment. The anchor device 100 can be used to anchor or secure a prosthetic mitral valve PMV deployed between the left atrium and left ventricle of a heart. The anchor device 100 can be used, for example, to anchor or secure the prosthetic mitral valve PMV via a tether 128 as described above with respect to FIG. 1. The anchor device 100 can also seal a puncture formed in the ventricular wall (not shown in FIG. 2) of the heart during implantation of the prosthetic mitral valve PMV. The anchor device 100 can also be used in other applications to anchor a medical device (such as any prosthetic atrioventricular valve or other heart valves) and/or to seal an opening such as a puncture.

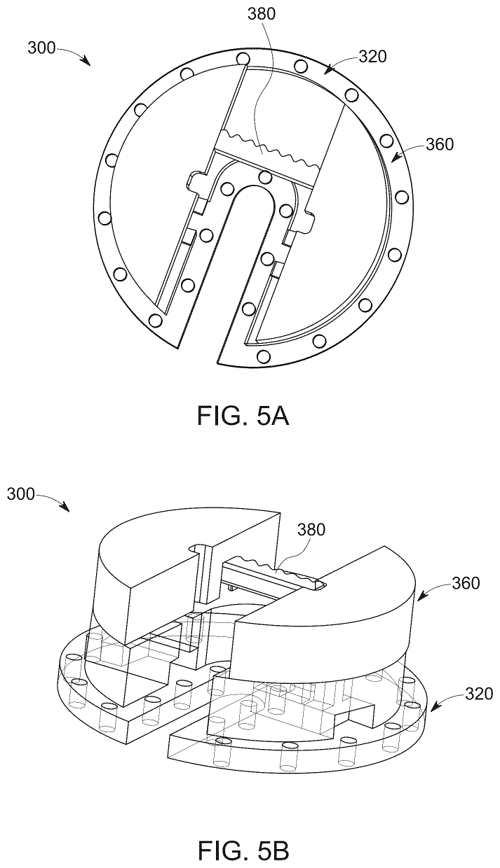

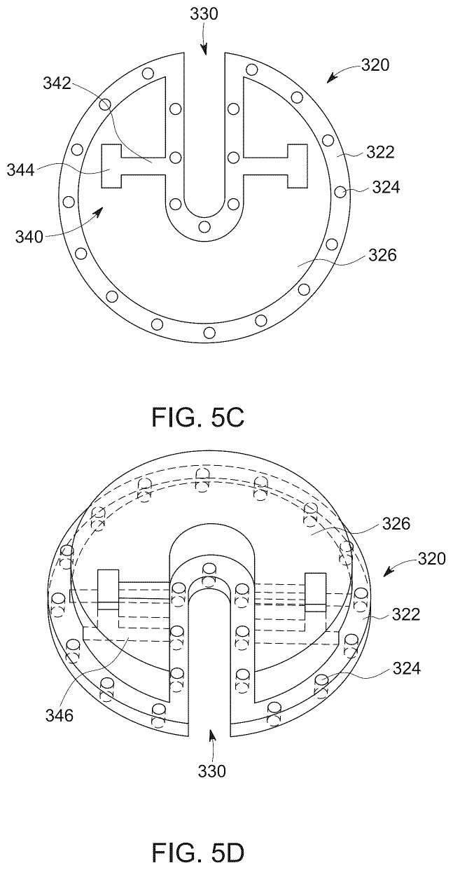

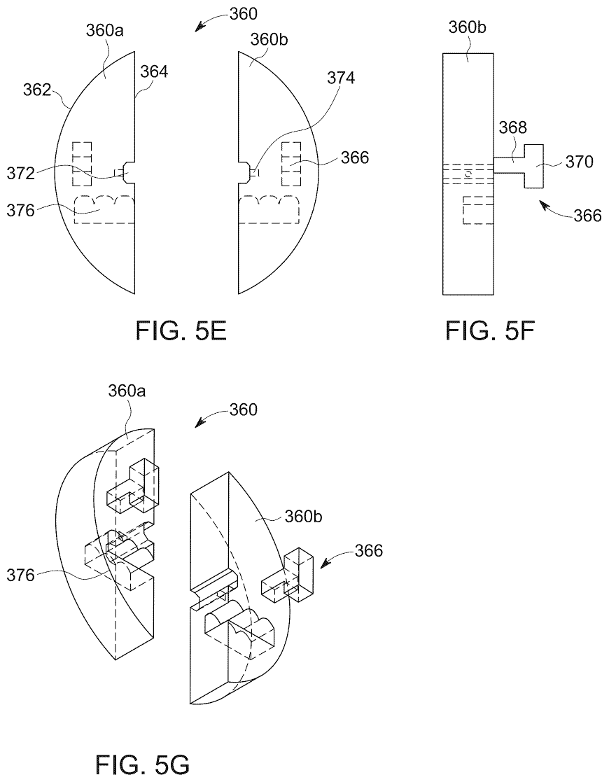

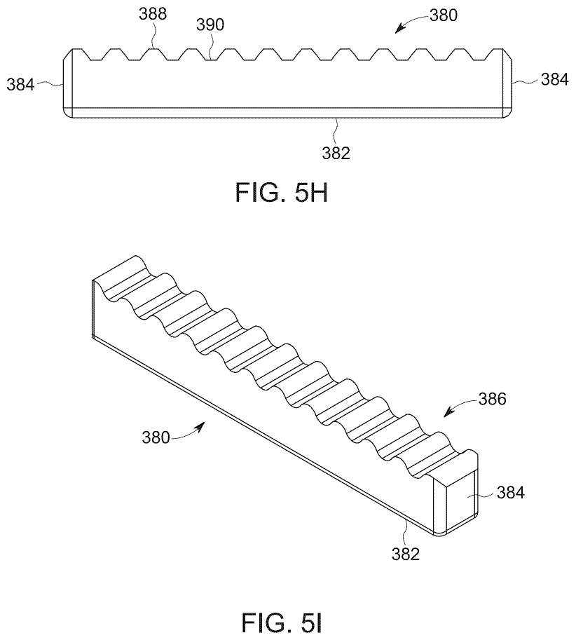

The anchor device 100 can include a pad (or pad assembly) 120, a tether attachment member 124, and a locking pin 126. The pad 120 can contact the epicardial surface of the heart and can be constructed of any suitable biocompatible surgical material. The pad 120 can be used to assist in the sealing of a surgical puncture (e.g., a transapical puncture at or near the apex of the left ventricle) formed when implanting a prosthetic mitral valve. In some embodiments, the pad 120 can include a slot that extends radially to an edge of the pad 120 such that the pad 120 can be attached to, or disposed about, the tether 128 by sliding the pad 120 onto the tether 128 via the slot. Such an embodiment is described below with respect to FIGS. 5A-I.

In some embodiments, the pad 120 can be made with a double velour material to promote ingrowth of the pad 120 into the puncture site area. For example, pad or felt pledgets can be made of a felted polyester and may be cut to any suitable size or shape, such as those available from Bard® as PTFE Felt Pledgets having a nominal thickness of between 2.5 mm and 3.0 mm, including for example 2.6 mm, 2.7 mm, 2.8 mm, or 2.9 mm. In some embodiments, the pad 120 can be larger in diameter than the tether attachment member 124. The pad 120 can have a circular or disk shape, or other suitable shapes.

The tether attachment member 124 can provide the anchoring and mounting platform to which one or more tethers 128 can be coupled (e.g., tied or pinned). The tether attachment member 124 can include a base member (not shown) that defines at least a portion of a tether passageway (not shown) through which the tether 128 can be received and pass through the tether attachment member 124, and a locking pin channel (not shown) through which the locking pin 126 can be received. The locking pin channel can be in fluid communication with the tether passageway such that when the locking pin 126 is disposed in the locking pin channel, the locking pin 126 can contact or pierce the tether 128 as it passes through the tether passageway as described in more detail below with reference to specific embodiments.

The locking pin 126 can be used to hold the tether 128 in place after the anchor device 100 has been tightened against the ventricular wall and the tether 128 has been pulled to a desired tension. For example, the tether 128 can extend through a hole in the pad 120, and through the tether passageway of the tether attachment member 124. The locking pin 126 can be inserted or moved within a locking pin channel such that it pierces or otherwise engages the tether 128 as the tether 128 extends through the tether passageway of the tether attachment member 124. Thus, the locking pin 126 can intersect the tether 128 and secure the tether 128 to the tether attachment member 124.

The tether attachment member 124 can be formed with a variety of suitable biocompatible materials. For example, in some embodiments, the tether attachment member 124 can be made of polyethylene, or other hard or semi-hard polymer, and can be covered with a polyester velour to promote ingrowth. In other embodiments, the tether attachment member 124 can be made of metal, such as, for example, Nitinol®, or ceramic materials. The tether attachment member 124 can be various sizes and/or shapes. For example, the tether attachment member 124 can be substantially disk-shaped.

In some embodiments, the tether attachment member 124 can include a hub that is movably coupled to the base member of tether attachment member 124. The hub can define a channel that can receive a portion of the locking pin (or locking pin assembly) 126 such that as the hub is rotated, the hub acts as a cam to move the locking pin 126 linearly within the locking pin channel. As with previous embodiments, as the locking pin 126 is moved within the locking pin channel, the locking pin can engage or pierce the tether 128 disposed within the tether passageway and secure the tether 128 to the tether attachment member 124.

In use, after a PMV has been placed within a heart, the tether extending from the PMV can be inserted into the tether passageway of the anchor device 100 and the tension on the tether attachment device can be adjusted to the desired tension. The anchor device 100 (e.g., some portion of the anchor device such as the tether attachment member 124, or the hub) can be actuated such that the locking pin 126 intersects the tether passageway and engages a portion of the tether disposed within the tether passageway, securing the tether to the tether attachment member. In some embodiments, prior to inserting the tether into the tether passageway, the anchor device 100 can be actuated to configure the anchor device 100 to receive the tether. In some embodiments, the anchor device 100 can be actuated by rotating a hub relative to a base member of the tether attachment member 124 such that the locking pin 126 is moved from a first position in which the locking pin is spaced from the tether passageway and a second position in which the locking pin intersects the tether passageway and engages or pierces the portion of the tether.

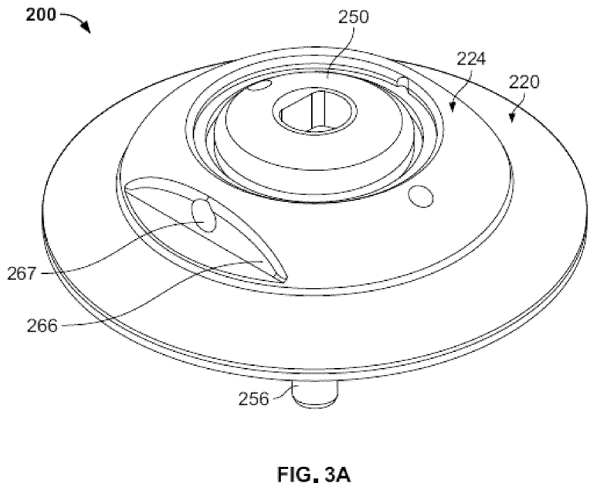

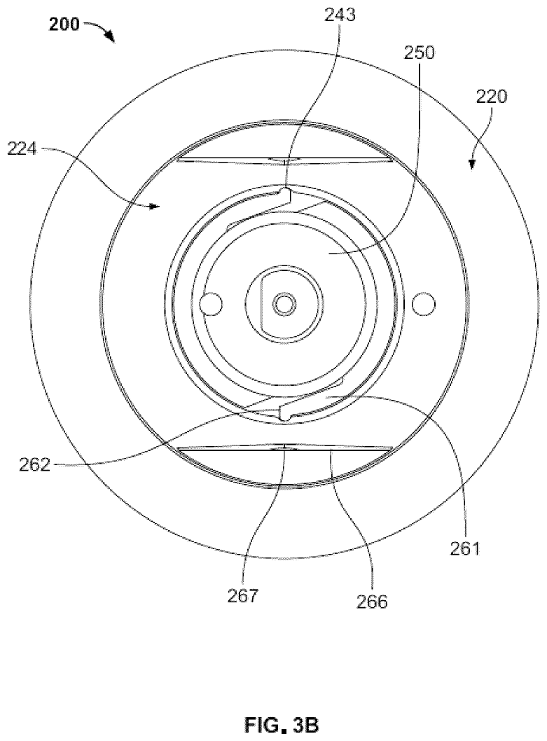

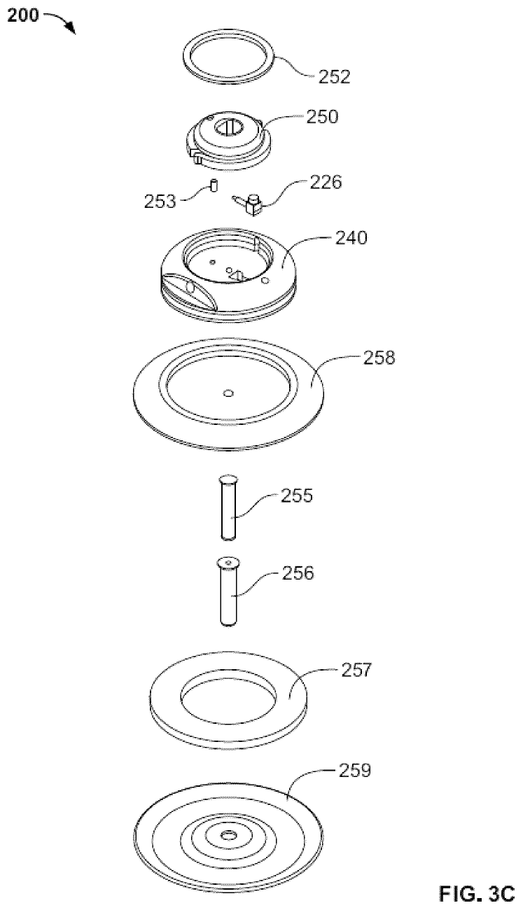

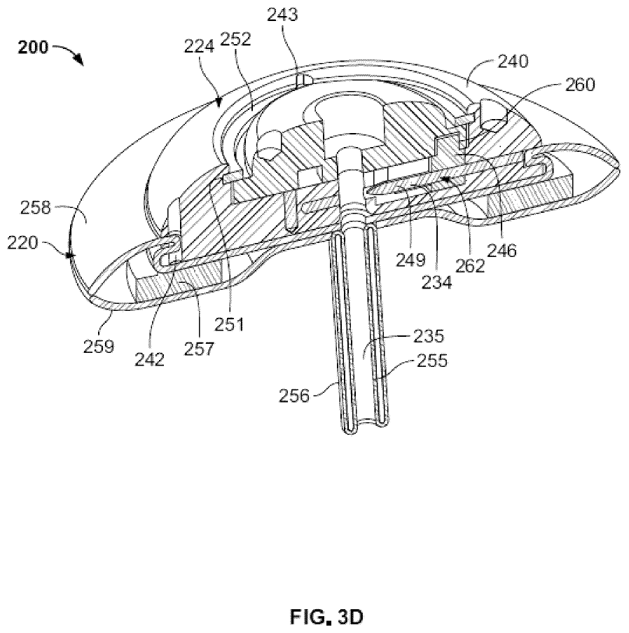

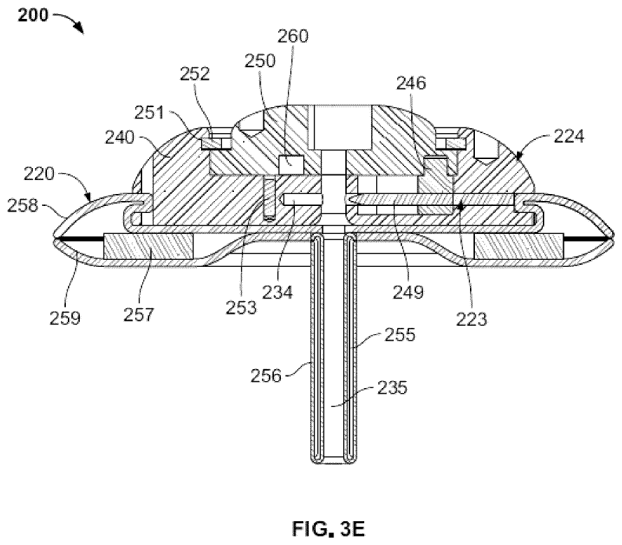

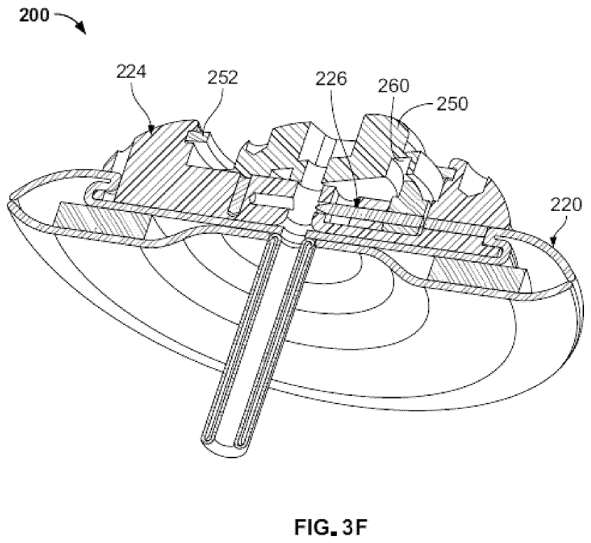

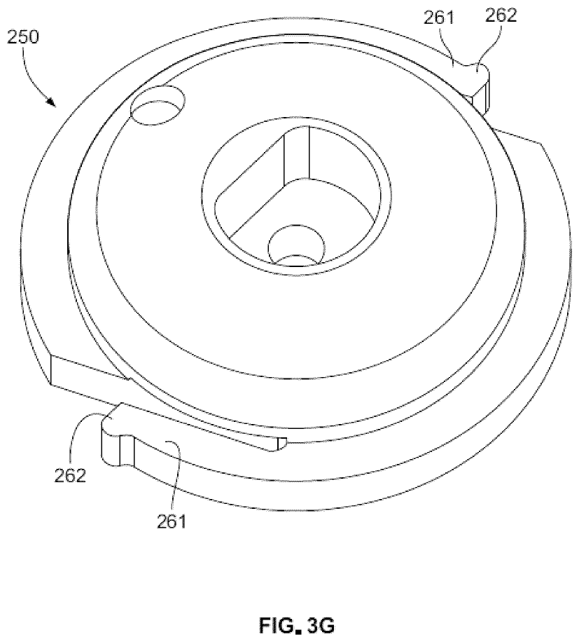

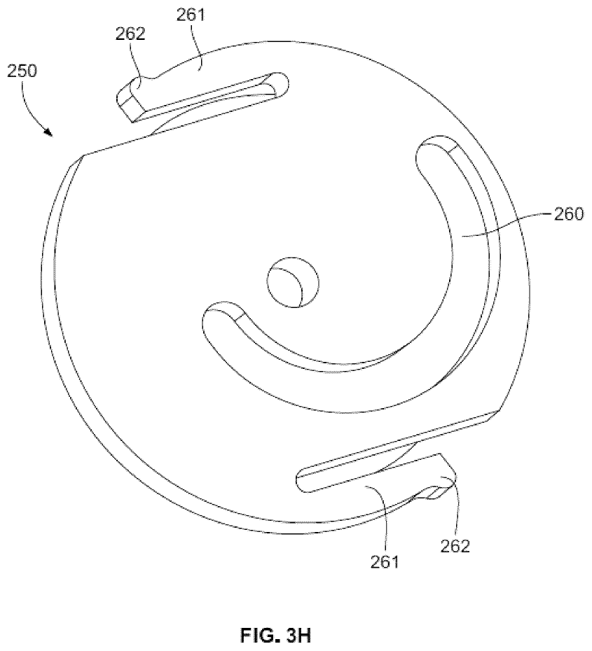

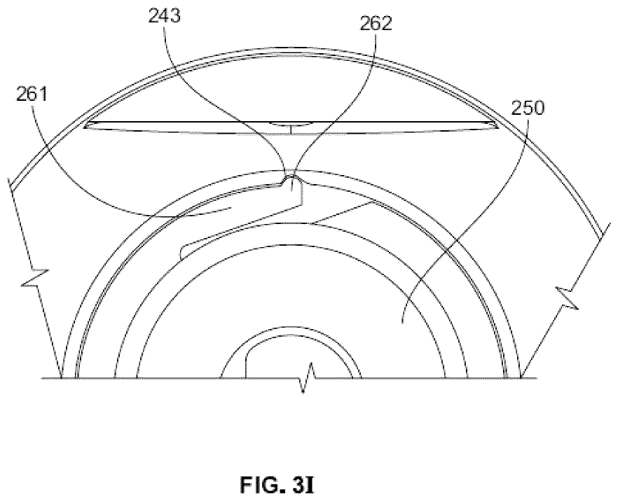

FIGS. 3A-I illustrate an epicardial anchor device according to another embodiment. An epicardial anchor device 200 includes a tether attachment member 224, a pad assembly 220, a tube member 255 and a tube cover member 256. The tether attachment member 224 includes a base member 240, a hub 250, a retaining ring 252, a locking pin assembly 226, and a pin member 253. The locking pin assembly 226 includes a driver portion 246 and a piercing portion 249. The base member 240 defines a circumferential pad channel 242, a retaining channel 251 and a locking pin channel 234. The pad channel 242 can be used to couple the pad assembly 220 to the tether attachment member 224. The retaining channel 251 can receive an outer edge of the retaining ring 252, which is used to retain the hub 250 to the base member 240. The base member 240 also defines cutouts or detents 243, as shown for example, in FIGS. 3B, 3D and 3I.

The tube member 255 is coupled to the base member 240 and the base member 240, the hub 250 and the tube member 255 collectively define a tether passageway 235 through which a tether (not shown) can be received. The cover member 256 can be formed with a fabric material, such as for example, Dacron®. The tether channel 235 intersects the locking pin channel 234 and is in fluid communication therewith.

The pad assembly 220 includes a top pad portion 258, a bottom pad portion 259 and a filler member 257 disposed therebetween. The top pad portion 258 and the bottom pad portion 259 can each be formed with, for example, a flexible fabric material. The top pad portion 258 and the bottom pad portion 259 can each define a central opening through which the tube member 255 can pass through. A portion of the top pad portion 258 is received within the channel 242 of the base member 240 as shown, for example, in FIGS. 3D-F.

An outer perimeter portion of the hub 250 is received within the retaining channel 251 such that the hub 250 can rotate relative to the base member 240 to actuate the locking pin assembly 226 as described in more detail below. As shown, for example, in FIGS. 3G and 3H, the hub 250 includes arms 261 with protrusions 262, which may also be referred to as detents or detent protrusions. The protrusions 262 can be received within cutouts 243 of the base member 240 and act as a stop or limit to the rotation of the hub 250. The slots 263 defined by the hub 250 enable the arms 261 to flex and allow the protrusions 262 to be moved in and out of the cutouts 243. As shown, for example, in FIGS. 3F and 3H the hub 250 defines a curved channel 260 on a bottom portion of the hub 250. The curved channel 260 is asymmetrical (or spiral) and receives the driver portion 246 of the locking pin assembly 226. As the hub 250 is rotated relative to the base member 240, the hub 250 acts as a cam to move the locking pin assembly 226 linearly within the locking pin channel 234. The locking pin assembly 226 can be moved from a first position in which the piercing portion 249 is disposed outside of the tether passageway 235 as shown in FIGS. 3D and 3E, and a second position in which the piercing portion 249 extends through the tether passageway 235 as shown in FIG. 3F. The pin member 253 (see, e.g., FIG. 3E) can be formed with a metal material that is more radio-opaque than the other components of the anchor device and thus visible to the user (e.g. physician) using conventional imaging modalities to enable the user to confirm that the locking pin assembly 226 has been fully moved to the second position.

In use, when the locking pin assembly 226 is in the first position, a tether (not shown) coupled to, for example, a prosthetic mitral valve and extending through a puncture site in the ventricular wall of a heart can be inserted through the tether passageway 235. The hub 250 can then be rotated 180 degrees to move the locking pin assembly 226 linearly within the locking pin channel 234 such that the piercing portion 249 extends through the tether passageway 235 and engages or pierces the tether, securing the tether to the tether attachment member 224. For example, when the locking pin is in the first position, the protrusions 262 of the hub 250 are each disposed within one of the cutouts 243 of the base member 240 (i.e., a first protrusion is in a first cutout, and a second protrusion is in a second cutout). The hub 250 can then be rotated 180 degrees such that the protrusions 262 are moved out of the cutouts 243 of the base member 240 and at the end of the 180 degrees the protrusions 262 are moved into the other of the cutouts 243 of the base member 240 (i.e., the first protrusion is now in the second cutout, the second protrusion is now in the first cutout).

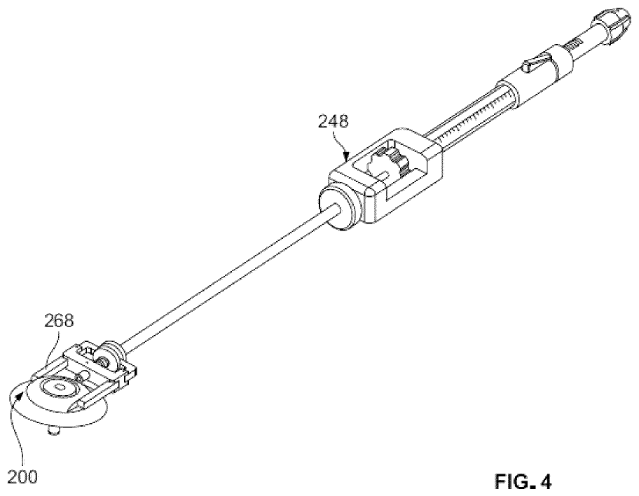

The base member 240 can also include cutout sections 266 and define side openings 267 (see, e.g., FIGS. 3A and 3B) that can be used to couple a delivery device to the epicardial anchor device 200. For example, FIG. 4 illustrates a delivery device 248 having coupling arms 268 and coupling pins (not shown) extending inwardly from the arms 268. The side openings 267 can receive the coupling pins and the cutout sections 266 can be engaged by the coupling arms 268.

In a typical use of epicardial anchor device 200 during a prosthetic mitral valve replacement, the prosthetic mitral valve is positioned within the patient's native mitral valve annulus through a transapical puncture, and a tether that is fixed to the prosthetic mitral valve extends through the transapical puncture so that it can be manipulated by a surgeon. The tether is passed through the tether passageway 235 and the epicardial anchor pad 200 is advanced over the tether into contact with the patient's heart. While the epicardial anchor pad 200 is in contact with the patient's heart, the tether is tensioned to the desired amount, for example using the delivery device 248, and then the tether is fixed at that desired tension using the pinning mechanism described in connection with epicardial anchor device 200. After the pinning is completed, and the surgeon is satisfied with the result of the implantation, the remaining length of the tether extending beyond the epicardial anchor device 200 is cut or otherwise trimmed to remove any excess length of tether that would otherwise remain in the patient's body. When the excess length of the tether is cut, it may be desirable to still leave about 5 cm of tether length to allow for future manipulation of the tether, if necessary. However, other lengths, such as about 1, 2, 3, 4, 5, 6, 7, 8, 9, or 10 cm, may be suitable. In some circumstances, after the excess length of the tether is cut, it may become necessary or desirable to further manipulate the epicardial anchor device 200 and/or the tether for various reasons, either immediately after cutting the excess length of the tether, or after days, weeks, months, or more have passed after the implantation procedure is completed. As noted above the tether is locked to the epicardial anchor device 200 at the desired tension during the initial implantation. In some circumstances, as time passes, the tension on the tether may decrease compared to the initially set tension. This may occur, for example, due to the anatomy changing, for example via ventricular remodeling (or shrinking) or otherwise acclimating to prosthetic mitral valve implant assembly. In some instances, the initial tension placed on the tether may be too small. Still further, it may be desirable to replace the first epicardial pad with another epicardial pad. In some circumstances, the ventricular tissue in contact with the epicardial pad may begin to dimple as a result of the force applied against the ventricular tissue by the epicardial pad. If the ventricle begins to dimple, and the epicardial pad sits within the dimpled area, the tension on the tether may decrease from the initially set tether tension. In this situation, it may be desirable to replace the first epicardial pad with a second epicardial pad that has a larger surface area of contact with the patient's heart compared to the first epicardial pad. By switching to an epicardial anchor pad with a relatively large surface area of contact with the patient's heart wall, the pressure on the heart tissue from the epicardial pad may be reduced, because the same tension force is applied over a larger surface area. The larger surface area may also prevent the larger epicardial pad from sitting within any dimpled area of the ventricular tissue. A reduction in pressure on the heart wall tissue may also reduce the likelihood of damage (or severity of damage) that may be caused on the heart wall tissue as a result of the contact force between the epicardial anchor device and the heart wall tissue. In other words, if dimpling occurred with a smaller epicardial pad, it may be less likely for dimpling to occur again after a smaller epicardial pad is switched out with a larger epicardial pad.

It may be difficult to switch out a first epicardial pad with a second epicardial pad, for example because it may be difficult to slide the original epicardial anchor device off the tether, and it may also be difficult to slide the new epicardial pad over the tether. For example, epicardial anchor device 200 has a central tether passageway 235 that would require the epicardial anchor device 200 to be slid proximally along the remaining length of the gripped tether to remove the epicardial anchor device, which may be practically difficult and/or time-consuming And if another different epicardial anchor device (e.g., one with a larger surface area of contact) is to replace the original pad, it may again be difficult to perform this replacement if the epicardial anchor device must be slid over the remaining length of tether being gripped outside of the patient's heart. In fact, even during the original placement of epicardial anchor device 200, it may be relatively complex and/or time-consuming to pass the end of the tether through the central aperture of the epicardial anchor device 200. Some of these challenges may be mitigated or eliminated by introducing certain design feature changes to the epicardial anchor device, as described below, particularly in connection with FIGS. 5A-I.

FIGS. 5A-B illustrate top and side perspective views, respectively, of an epicardial anchor device 300 according to an aspect of the disclosure. Anchor device 300 may generally include a base component 320, which may be configured to directly contact the patient's heart (e.g., at or near the exterior of a ventricular apex), a top component 360, and a rail 380. In FIGS. 5A-B, the base component 320 is shown in partial phantom. As will become clear from the description below, the anchor device 300 is shown in an unlocked or unpinned condition in FIGS. 5A-B.

FIGS. 5C-D are top and perspective views, respectively, of the base component 320 of anchor device 300. In FIG. 5D, the base component 320 is shown in partial phantom. Base component 320 may include a rim 322 that follows or defines a perimeter of the base component 320, the perimeter being generally circular but for a generally “U”-shaped section that defines a portion of a slot 330, described in greater detail below. The rim 322 may have a small thickness compared to the thickness of the entire base component 320, and may include a plurality of apertures 324 spaced around the perimeter of the rim 322. A fabric having a shape that generally matches the bottom surface of the base component 320 (i.e., the surface that will face the patient's heart when in use) may be placed into contact with the bottom surface of the base component 320 and coupled to the base component 320 via sutures that pass through the apertures 324. The fabric may help to seal the puncture in the heart and to establish ingrowth after implantation. However, in some embodiments, the fabric may either be omitted or coupled to the base component 320 by other means than sutures that pass through apertures 324.

Still referring to FIGS. 5C-D, the base component 320 may include a main body 326 that extends upward away from the bottom surface of the base component 320. The main body 326 may follow the same general contour as the rim 322, with the rim 322 extending from the main body 326. The main body 326 may be generally circular but for a generally “U”-shaped section that defines a portion of the slot 330. In addition, the main body 326 may include two slots 340 that are substantially identical to each other but having mirrored or symmetric orientations about a centerline that bisects slot 330. The top of each slot 342 may include a narrow portion 342 that includes a first side that opens toward slot 330, and a second opposite end that transitions into a wide portion 344. The wide portion 344 may be enclosed by the main body 326 opposite the area where it transitions to narrow portion 342. This configuration, when viewed from the top as best shown in FIG. 5C, results in two “T”-shaped slots, with the narrowed portion of each “T”-shaped slot pointing to each other. As best shown in the partial phantom view of FIG. 5D, the bottom of each slot 340 may end in alignment with the rim 322, so that the bottom surface of the base component 320 is completely closed, but for the apertures 324 and slot 330. Each slot 340 may define a bottom portion 346 that had a width that is about equal to the width of the wide portion 344. In other words, although the slots 340 have a narrowed portion 342 at the top, the slots 340 may be wider underneath the narrowed portions 342. As described below, this configuration allows for “T”-shaped projections 366 of the top component 360 to be inserted into the wide portion 344 of the slot 340, and to slide along the narrowed portion 342 without risk of the top component 360 decoupling from the base component 320.

FIGS. 5E, 5F, and 5G show top, side, and perspective views, respectively, of the top component 360. Top component 360 may include two substantially identical pieces 360a, 360b, with each piece being substantially a mirror image of the other. Thus, the description of either piece 360a, 360b is applicable to the other piece. Each piece 360a, 360b may have an outer contour 362 that is substantially an arc of a circle and a generally straight inner edge 364. When the top component 360 is assembled to the base component 320, the inner edges 364 may face each other and the outer contours 362 may face away from each other. Further, when assembled and in the particular unpinned condition shown in FIGS. 5A-B, the outer contours 362 may generally align with corresponding contours of the main body 326 of the base component 320. As best shown in the side view of FIG. 5F, each piece 360a, 360b may include a generally “T”-shaped protrusion 366 extending from a bottom surface thereof, the protrusion 366 including a narrowed portion 368 coupled to the piece 360a, 360b and a wider terminal portion 370. As should be understood from the description above, the protrusion 366 of each piece 360a, 360b may be inserted into a corresponding wide portion 344 of a slot 340 in the base component 320 to couple each piece 360a, 360b to the base component 320. Then, the two pieces 360a, 360b may slide (e.g., upon application of manual force) toward or away from each other, with the wide portion 370 of each protrusion 366 sliding along the bottom portion 346 of the slot 340. With this configuration, lateral motion of the pieces 360a, 360b is permitted, but the larger side of the wide portion 370 of each protrusion 366 compared to the smaller size of the narrow portion 342 of the slot 340 prevents either piece 360a, 360b from pulling away from and decoupling from the base component 360. Preferably, when the two pieces 360a, 360b are at their closest distance to each other (e.g., having been manually pushed together so that the flat edges 364 touch or nearly touch), the wide portion 370 of each protrusion 366 still remains adjacent to the narrow portion 342 of the slot 340 to prevent decoupling.

As best shown in FIGS. 5E and 5G, the inner straight edge 364 of each piece 360a, 360b may be interrupted by a notch or recess 372, which may be positioned at about the center of the straight edge 364. Each recess 372 may have the shape of a semi-circle, an arc, a general “U” or “C”-shape, or the like. When the pieces 360a, 360b are pressed toward each other so that the straight edges 364 touch or nearly touch, the recesses 372 align with each other to create an aperture with closed (or substantially closed) sides, but being open through the top and bottom of the top component 360. From each recess 372, a small channel 374 may extend away from the straight edge 364 and away from the recess 372 toward the contoured outer side 362, the channel 374 only opening to the recess 372. A pin may be fixed (e.g., via adhesive) in one of the channels 374, with the pin having a generally sharp end that faces away from the channel 374 in which it is received. When the two pieces 360a, 360b are pressed toward each other so that the straight edges 364 touch or nearly touch, the sharp end of the pin traverses the aperture formed by the two recesses 372, and extends into the channel 374 of the opposite piece 360a or 360b. As will be described in greater detail below, the aperture formed by the recesses 372 is sized and shaped to receive the tether therethrough, with the pin piercing the tether when the pieces 360a, 360b are pressed together into the pinned condition.

As best shown in FIGS. 5E and 5G, each piece 360a, 360b may include a rail channel 376. In the illustrated embodiment, each rail channel 376 is open only to the flat edge 364, with the rail channel 376 extending in a direction within the piece 360a, 360b toward the outer contoured portion 362. The rail channels 376 are aligned with each other so that, when the two pieces 360a, 360b are pressed together so that the flat edges 364 touch or nearly touch, the two separate rail channels 376 form a single continuous rail channel. The rail channel 376 may have a shape that corresponds to the shape of the rail 380 that is to be received therein. As best shown in FIGS. 5H-I, the rail 380 may have the general shape of a rectangular prism, with a substantially flat bottom surface 382 that extends to two side surfaces 384. The top surface 386 may have a plurality of bumps or notches. In the illustrated embodiment, the top surface 386 includes a plurality of peaks 388 alternating with a plurality of troughs 390 in a direction between the two side surfaces 384. Referring back to FIGS. 5E and 5G, the rail channels 376 may have a generally complementary shape, with a top surface that defines the rail channels 376 including alternating peaks and troughs. The rail 380 may be formed of a material that has at least a slight deflection capability at the peaks 388. With this configuration, when a first end of the rail 380 is received within a rail channel 376 of piece 360a and a second end of the rail 380 is received within a rail channel 376 of piece 360b (as shown in FIGS. 5A-B), the peaks 388 that are received within a rail channel 376 will snap or lock with a corresponding trough of the rail channel 376. In the absence of intentional applied force, the interaction between the rail 380 and the rail channels 376 will prevent the two pieces 360a, 360b from moving toward or away from each other. However, upon an intentional application of pulling or pushing force, the peaks 388 received with the rail channels 376 may slightly deflect or deform to allow for the pieces 360a, 360b to move toward or away from each other until the peaks 388 again align with the next set of troughs of the rail channels 376. With this configuration, pieces 360a, 360b will maintain their relative position until and unless a threshold force is applied to press the pieces 360a, 360b together or to pull the pieces 360a, 360b apart, with the threshold force being large enough to cause deflection or deformation of the peaks 388 of the rail 380. The total length of the rail 380 is preferably about equal to the combined length of the two rail channels 376 so that the rail 380 does not prevent the pieces 360a, 360b from being pressed close together into a condition in which the straight edges 364 touch or nearly touch. However, the total length of the rail 380 is preferably long enough so that, when the pieces 360a, 360b are separated and the anchor device 300 is in the unpinned condition shown in FIGS. 5A-B, at least one peak 388 is received within a corresponding trough of each rail channel 376. It should be understood that other specific designs of the rail 380 (and corresponding designs of the rail channels 376) may be implemented to achieve substantially the same effect, and the particular design illustrated in FIGS. 5E-51 is merely exemplary. Even if the rail 380 has the same design as shown herein, the rail channels 376 may have other designs. For example, the rail channels 376 may include spring and ball detent mechanisms, such that the spring presses the ball into a trough or other corresponding detent in the rail 380, with the force being able to be overcome via manual adjustment of the pieces 360a and/or 360b. In other examples, instead of the rail channels 376 including a plurality of troughs and peaks, each rail channel 376 may include a singular notch. In other words, although one specific configuration of rail channels 376 is shown, other designs may suitably achieve the same effect as the illustrated embodiment.

In use, a prosthetic mitral valve PMV may be implanted as described above such that, while the prosthetic mitral valve PMV is seated within the native valve annulus, a tether coupled to the prosthetic mitral valve extends through a wall of the heart, for example at the left ventricular apex, so that the tether is accessible to the surgical personnel. Instead of needing to thread or otherwise pass an end of the tether through a closed aperture (e.g., as is the case with anchor device 200), the tether may be laterally slid through the slot 330 of the anchor device 300 until the tether is near the end of the slot, which may be near a radial center of the anchor device 300. During this part of the procedure, the two pieces 360a, 360b of the top component are spaced apart in the unpinned condition, similar to the configuration shown in FIGS. 5A-B, so that the two pieces 360a, 360b do not interfere with sliding the tether radially inwardly along the slot 330. Further, as described above, the rail 380 allows the two pieces 360a, 360b to maintain this open position without any substantial likelihood of unintentional movement of the pieces 360a, 360b toward each other. Once the tether is positioned at or near the end of the slot 330 and the tether is tensioned to the desired amount, the user may manually press pieces 360a, 360b toward each other, ensuring that the tether is aligned with the two recesses 372 that are moving toward each other. The user continues to move piece 360a toward 360b, overcoming the threshold force required to allow the rail 380 to deflect or deform, until the two recesses 372 form an aperture capturing the tether, with the pin having pierced the tether and the free, sharp end of the pin being received in the corresponding channel 374. At this point, the two flat edges 364 of the pieces 360a, 360b are in contact (or nearly in contact) with each other, the rail 380 is maintaining the two pieces 360a, 360b in the desired lateral position relative to each other, and the tether is pinned by the pin and secured within the aperture defined by the adjoining recesses 372. Further, in this position, the bottom surface of the anchor device 300, which preferably includes a fabric thereon, is pressed against the wall of the heart and preferably applying pressure to the incision previously formed in the wall of the heart. Thus, the anchor device 300 interacts with the tether to maintain the tether at the desired tension, which may provide stability to the prosthetic mitral valve PMV within the native annulus, while also helping to stop any bleeding at the incision site. If the user needs to adjust the tether for any reason, the user may pull the pieces 360a, 360b apart to unpin and adjust the tension on the tether (and/or to release the tether), and if necessary may slide the anchor device 300 laterally away from the tether, without needing to slide the tether through a small hole.

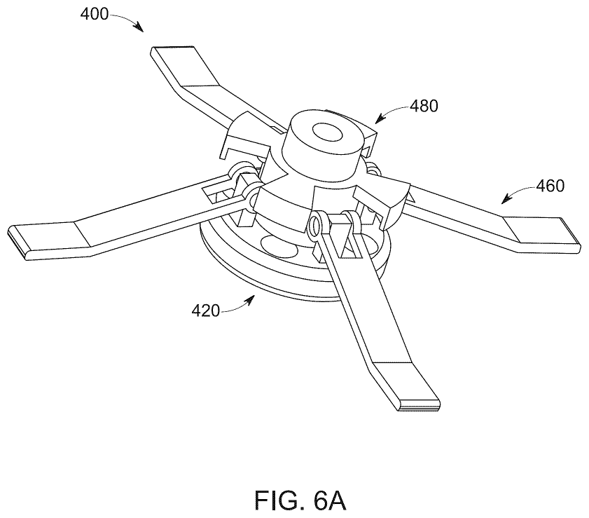

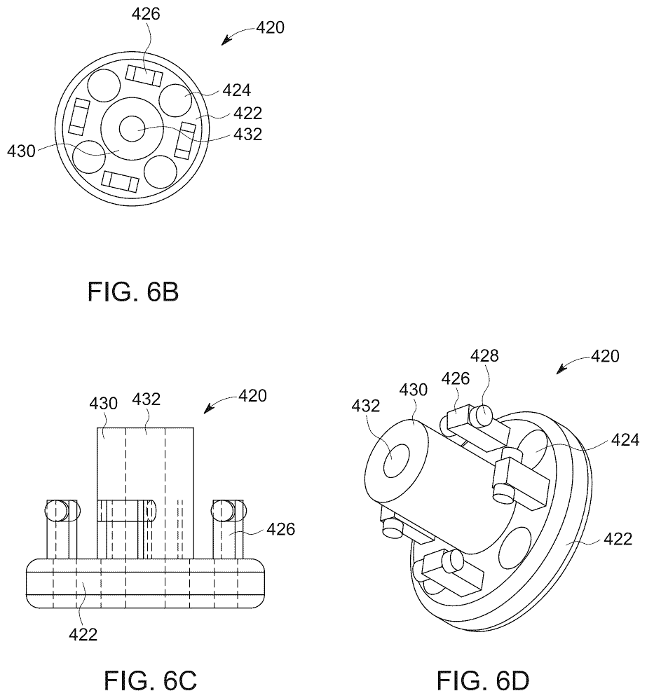

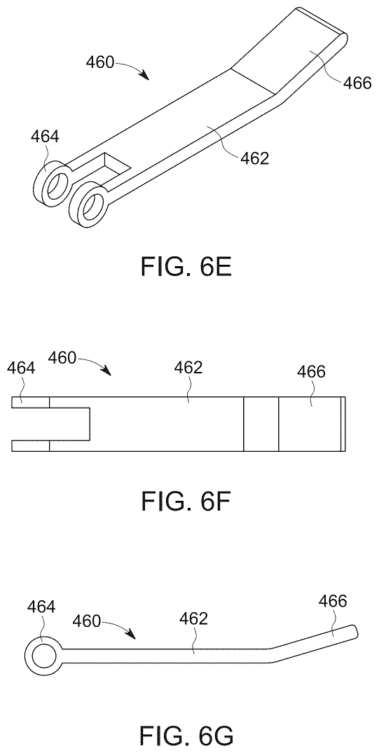

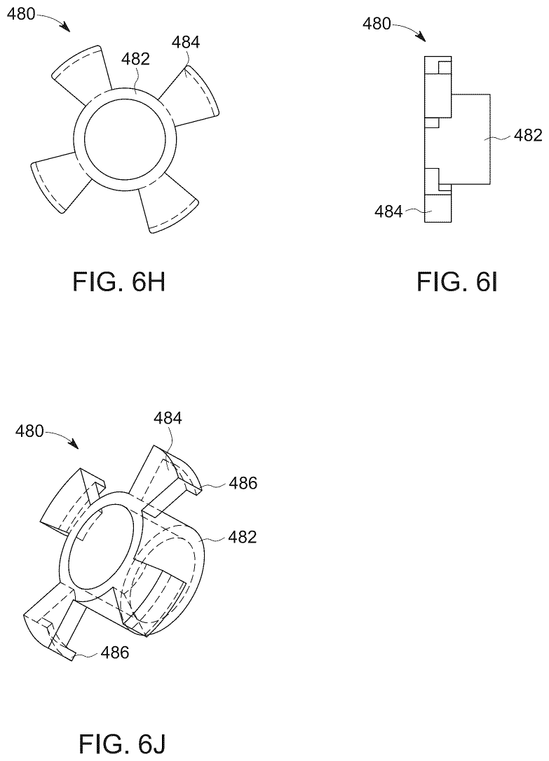

As described above, when using an epicardial anchor device that is generally rigid and/or non-collapsible, the size of the incision required to pass the anchor device through the patient and into contact with the patient's heart is generally closely related to the size of the anchor device itself. It is generally desirable to minimize the size of the incision required to implant a prosthetic mitral valve PMV and an associated epicardial anchor device, but it is important that the effectiveness of the anchor device is not reduced as a result of a design change to reduce the size of the anchor. The epicardial anchor device 400 described in connection with FIGS. 6A-J may allow for a relatively small incision to allow the anchor device to pass into the patient and into contact with the patient's heart, without reducing the effectiveness of the anchor.

FIG. 6A is a perspective view of epicardial anchor device 400 in an expanded, actuated, or deployed condition. Generally, epicardial anchor device 400 may include a base component 420 that is intended to contact an outer surface of the patient's heart, a plurality of hinged arms 460 that may be stowed for delivery or actuated or deployed (as shown in FIG. 6A) to provide additional contact with the patient's heart, and a top component or cover 480 for locking the hinged arms 460 in the deployed condition. In the assembled condition shown in FIG. 6A, the components of the anchor device 400 may define a central passageway (which may be mostly or entirely defined by the base component 420) that allows for passage of the tether.

Base component 420 is shown in FIGS. 6B-D. Base component 420 may include a base 422 which may be generally circular and intended to contact the heart (including indirect contact via a fabric interposed between the base component 420 and the heart tissue). The base component 420 may have a diameter (e.g., in the view of FIG. 6B) that is significantly smaller than the diameter of anchor devices 200 or 300. As should be understood from the description below, the smaller diameter of the base component 420 may allow for a smaller incision site (and may reduce the need to spread the patient's ribs and/or reduce the extent of rib-spreading needed and thus reduce the likelihood of breaking ribs). However, despite the smaller diameter of the base component 420 resulting in a smaller area of contact with the patient's heart, additional contact with the heart via the hinge arms 460 may result in sufficient distribution of forces to the heart.

Still referring to FIGS. 6B-D, the base 422 may include a plurality of apertures 424 extending therethrough. As with apertures 324, apertures 424 may be used to couple a fabric (e.g., via sutures) or other member configured to promote tissue ingrowth and/or assist with sealing the puncture site of the heart. Base 422 may also include a plurality of hinge posts 426 extending upwardly from the base 422 (away from the bottom surface of the base 422 which is intended to contact tissue of the patient's heart). In the illustrated embodiment, the base 422 includes four hinge posts 426 at equal intervals around the base 422 (e.g., every 90 degrees), with an aperture 424 positioned between each adjacent pair of apertures 424. The number of hinge posts 426 is preferably the same as the total number of hinge arms 460 of the anchor device 400, which may be four, but may be more or fewer than four depending on the desired load distribution. Preferably, each hinge post 426 does not extend radially outward of the outer diameter of the base 422. In the illustrated embodiment, each hinge post 426 includes two opposing round protrusions 428 that are configured to engage corresponding loops 464 of the hinge arms 460 to allow the hinge arms 460 to rotate about the connection to the hinge posts 426. However, other mechanisms besides loops 464 and protrusions 428 may be used to provide the desired hinged motion between the hinge arms 460 and the hinge posts 426.

Still referring to FIGS. 6B-D, the base component 420 may include a central post 430 extending upwardly from the base 422, preferably to a height that is greater than the heights of the hinge posts 426. The central post 430 may be generally cylindrical and may include a central apertures or channel 432 extending through the central post 430 and also through the base 422. In the illustrated embodiment, the central post 430 is positioned radially inwardly of the series of apertures 424 and hinge posts 426. As is described in greater detail below, the central post 430 may serve, at least in part, as a support for the cover 480 to assist with locking the hinge arms 460 in the deployed condition. The connection between the hinge arms 460 and the hinge posts 426 is shown in FIG. 6A. Referring back to FIGS. 6E-G, an angled portion 466, which may still be a generally flat rectangular shape, extends from the opposite end of the main body 462.

An exemplary hinge arm 460 is shown in FIG. 6E. In the particular example shown, each hinge arm may include a generally rectangular flat main body 462. On a first end of the main body 462, two loops 464 may extend from opposite ends of the main body 462, the spacing between the pair of loops 464 being configured to receive the hinge post 426 therebetween while the protrusions 428 are received within corresponding loops 464. As is described in greater detail below, when the hinge arms 462 are deployed, the angled portions 466 may be generally parallel to the bottom of the base 422, so that both the bottom of the base 422 and the bottom face of the angled portions 466 all contact the heart tissue to better distribute the load from the force of the tensioned tether. In some embodiments, the bottom faces of the angled portions 466 may include a fabric or similar feature to enhance tissue ingrowth after prolonged contact with the tissue of the heart. When the hinge arms 460 are deployed, as shown in FIG. 6A, the hinge arms 460 extend radially outward of the diameter of the base component 420. However, the hinge arms 460 may be rotated upwardly toward the central post 430 of the base component 420 for delivery. When rotated upwardly in a generally vertical condition, the hinge arms 460 do not extend radially outwardly beyond the diameter of the base 422 of the base component 420. In other words, when the anchor device 420 is in the stowed or delivery condition with the hinge arms 460 raised, the largest profile of the anchor device is the diameter of the base 422 of the base component 400. It should be understood that, while one exemplary geometric configuration of hinge arms 460 is shown in FIGS. 6E-G, other configurations may also be suitable.

FIGS. 6H-J illustrate different views of an embodiment of a cover 480 configured to lock the hinge arms 460 in the deployed condition. In the illustrated embodiment, the cover 480, which may also be referred to as a top component or hinge arm lock, includes a generally tubular portion 482 having an inner aperture sized and shaped to receive the central post 430 of the base component 420 therethrough (as shown in FIG. 6A). The cover 480 may include a plurality of radial extensions 484 extending radially outwardly from the tubular portion 482. In the illustrated example, radial extensions 484 are each generally fan shaped, although other shapes may be suitable. In the illustrated embodiment, a total of four radial extensions 484 are included, but it should be understood that if more or fewer than four hinge arms 460 are provided, a corresponding larger or smaller number of radial extensions 484 would preferably be used. The radial extensions 484 preferably have a generally equivalent circumferential spacing as the hinge posts 426. Thus, in the illustrated embodiment, the radial extensions 484 are positioned at about 90-degree intervals. Furthermore, the radial extensions 484 are preferably sized so that there is a gap or recess between each adjacent pair of radial extensions 484, with the gap large enough to accommodate the width of a hinge arm 460. Preferably, the radial extensions 484 are positioned at the top end of the tubular portion 482, so that when the cover 480 is assembled to the central post 430 the radial extensions are generally positioned just above the hinge posts 426. In some embodiments, each radial extension 484 may include a lip 486 extending downwardly from the terminal or free end of the radial extension 484. The radial extensions 484 may be sized so that the lips 486 are positioned just radially outwardly of the hinge posts 426.

Referring to FIG. 6A, the cover 480 may have generally two functional rotational positions, including the unlocked position shown in FIG. 6A. In this unlocked position, the gap between adjacent radial extensions 484 is aligned with each hinge arm 460 so that the hinge arms 460 are free to rotate upwardly to be positioned through the gap, or to be rotated downwardly to the deployed condition as illustrated. If the cover 480 is rotated to the second locked position (which would be about 45 degrees of rotation when four hinge arms 460 and radial extensions 484 are included), the extensions 484 overlie the hinge arms 426 and the lips 486 extend downwardly to contact (or nearly contact) the top surface of the main body 462 of the hinge arms 460. When in this locked position, the hinge arms 460 are not able to be rotated upwardly into the vertical condition because the extensions 484 and the lips 486 mechanically prevent upward rotation of the hinge arms 460. In some embodiments, the cover 480 may be connected to the central post 430 with a mechanism to assist with rotation and/or to passively lock the cover 480 in either the locked position or the unlocked position. For example, the cover 480 may be threadedly coupled to the central post 430 to prevent undesired rotation. In other embodiments, the cover 480 may include inwardly extending protrusions and the central post 430 may include divots or grooves (or vice versa) to passively lock the cover 480 in the locked and unlocked conditions. Still other coupling mechanisms, such as tongues and grooves, may be provided to create similar functionality.

In use, a prosthetic mitral valve PMV may be implanted as described above such that, while the prosthetic mitral valve PMV is seated within the native valve annulus, a tether coupled to the prosthetic mitral valve extends through a wall of the heart, for example at the left ventricular apex, so that the tether is accessible to the surgical personnel. The anchor device 400 may be placed in the stowed condition by rotating the hinge arms 460 upwardly to be positioned in the gap between circumferentially adjacent radial extensions 484. While in this stowed condition, the anchor device 400 may only need clearance of the diameter of the base 422 of the base component 420. As a result, the incision site may be relatively small, and rib spreading may not be needed (or may only be minimally needed). The free end of the tether may be threaded or otherwise passed through the central lumen 432 of the base component, with the anchor device 400 being slide along the tether toward the patient's heart, with the bottom surface of the base 422 facing the heart.

Once the bottom face of the base 422 is in contact with the tissue of the heart, the tether may first be tensioned and locked at the desired tension, and then the hinge arms 460 deployed and locked. However, these two steps may be performed in the opposite order if desired. The tether may be locked at the desired tension via any suitable mechanism, including for example a clamping mechanism operably coupled to the anchor device 400, a pinning mechanism within the central post 430, or any other suitable mechanism. Other suitable mechanisms are described in U.S. Provisional Patent Application No. 63/342,801 filed May 17, 2022, and titled “Fully-Transseptal Cinching Apical Pad,” the disclosure of which is hereby incorporated by reference herein. Regardless of the order of operations, in order to actuate or deploy the hinge arms 460, the hinge arms 460 may be rotated toward the heart until the angled portions 466 contact the heart tissue. Because the hinge arms 460 are already through the incision site at this point, a larger incision can be avoided because the hinge arms 460 are passed through the incision site while stowed in the small profile condition. Once the hinge arms 460 are deployed, the user may rotate the cover 480, for example 45 degrees, until the radial extensions 484 overlie the hinge posts 426, which prevent the hinge arms 460 from tending to rotate back toward the stowed condition. Thus, despite the base 422 of the base component 420 having a small profile, there is still ultimately a large surface area of contact with the heart due to the additional areas of contact between the heart tissue and the angled portions 466 of the hinge arms 460. If the tensile force from the tether pulling the anchor device 400 is high and the surface area of contact with the heart tissue is small, dimpling in the heart tissue may result which may be undesirable as described above.

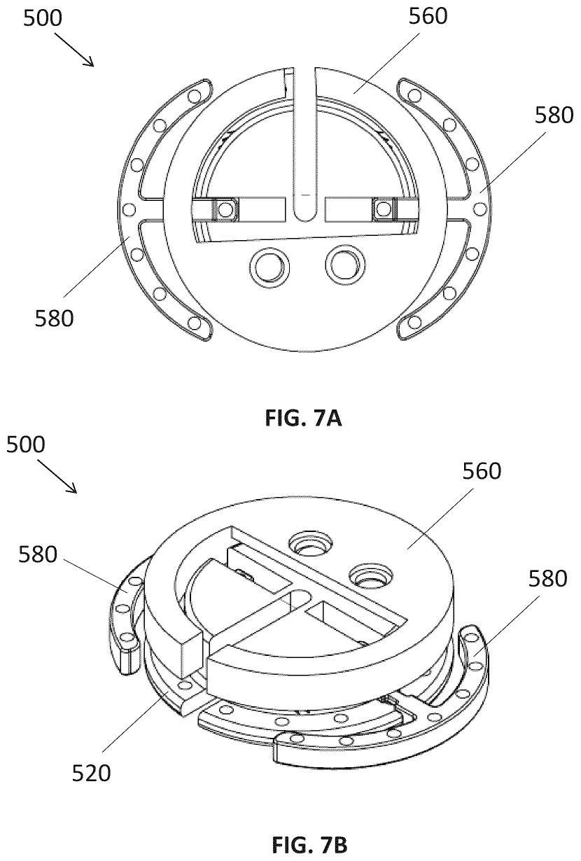

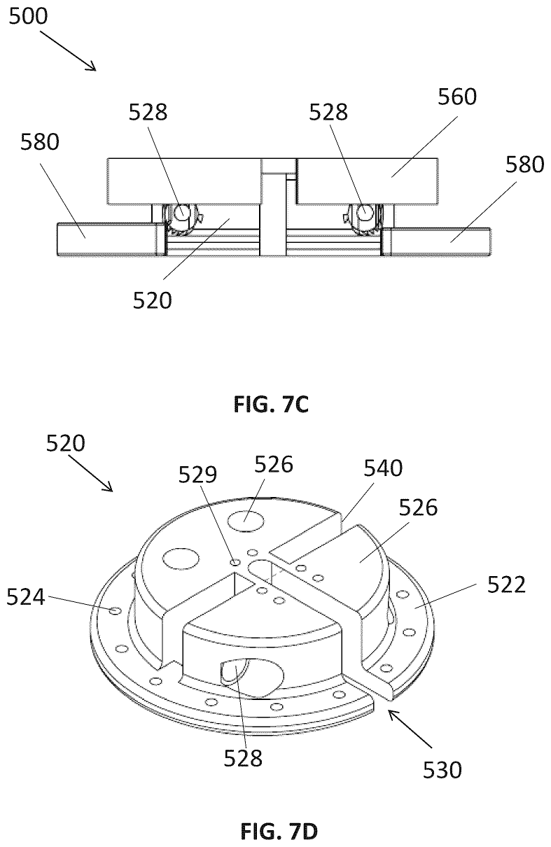

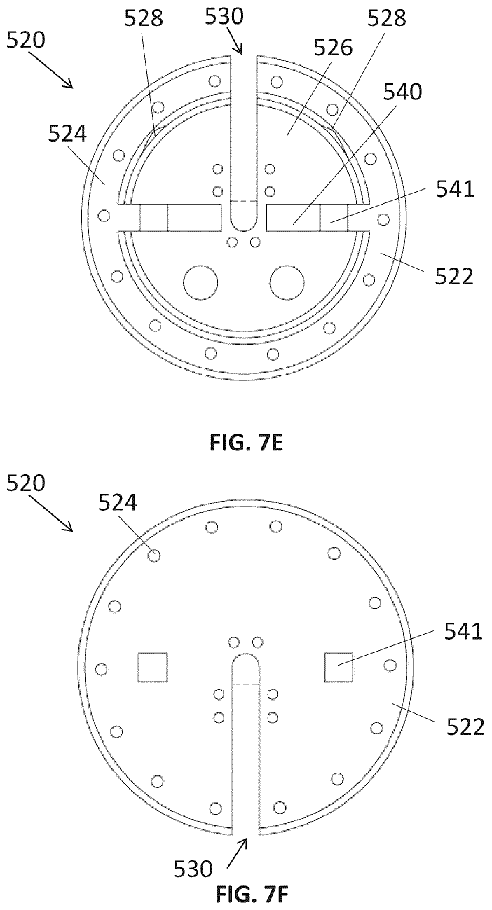

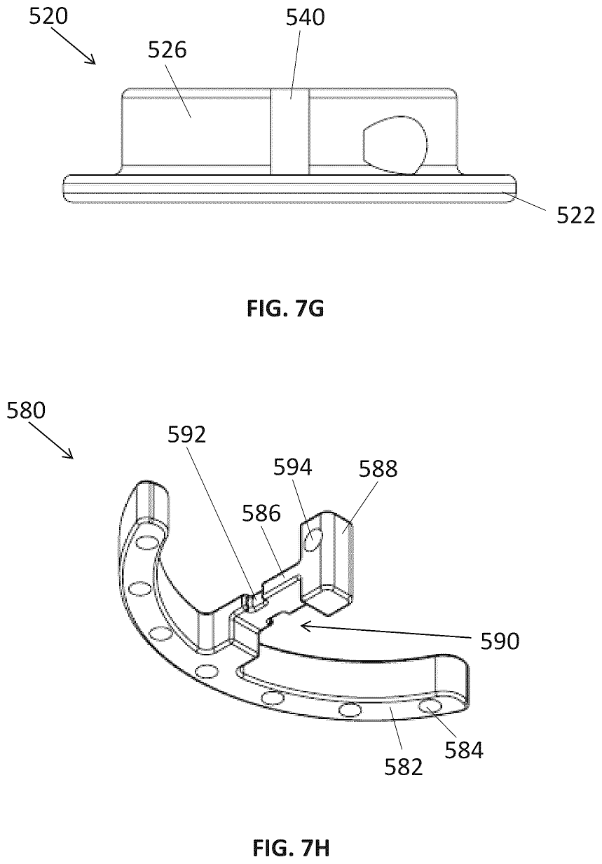

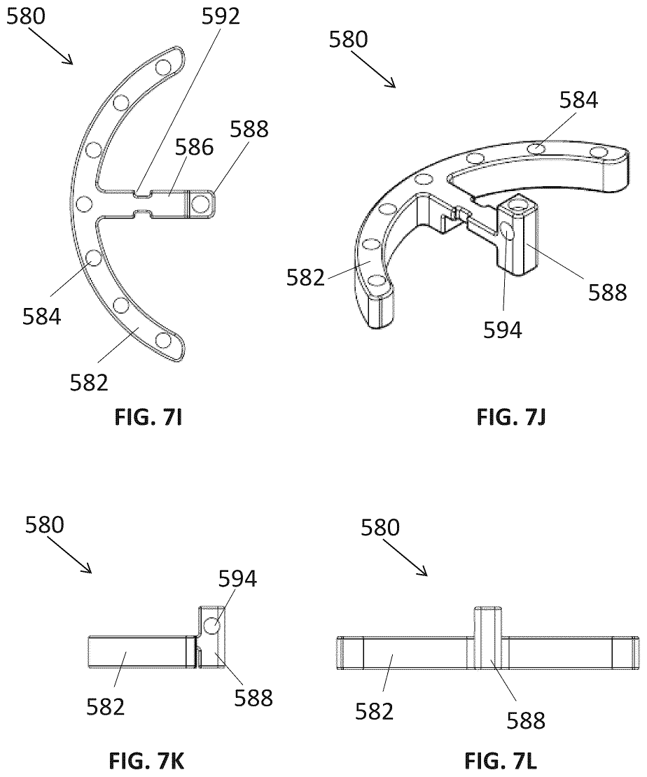

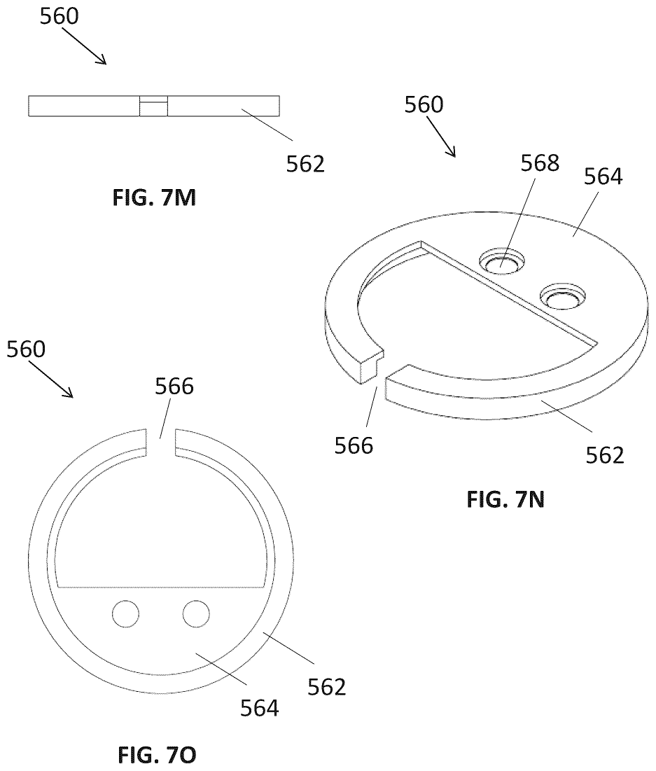

FIG. 7A-C are top, perspective, and side views, respectively, of an epicardial anchor device 500 according to another aspect of the disclosure. Anchor device 500 may generally include a base component 520, which may be configured to directly contact the patient's heart (e.g., at or near the exterior of a ventricular apex), a cap 560, and a pair of extension arms 580. In FIGS. 7A-C, the anchor device is shown in an expanded condition with extension arms 580 in an extended position. However, as should become clear, the extension arms 580 may be pressed radially inwardly from the condition shown in FIGS. 7A-C to place the anchor device 500 in a contracted position in which the outer diameter of anchor device is about equal to the outer diameter of the base 520 and/or cap 560.

FIGS. 7D-G are perspective, top, bottom, and side views, respectively, of the base component 520 of anchor device 500. Base component 520 may include a rim 522 that follows or defines a perimeter of the base component 520, the perimeter being generally circular but for a generally “U”-shaped section that defines a portion of a slot 530, described in greater detail below. The rim 522 may have a small thickness compared to the thickness of the entire base component 520, and may include a plurality of apertures 524 spaced around the perimeter of the rim 522. A fabric having a shape that generally matches the bottom surface of the base component 520 (i.e., the surface that will face the patient's heart when in use) may be placed into contact with the bottom surface of the base component 520 and coupled to the base component 520 via sutures that pass through the apertures 524. The fabric may help to seal the puncture in the heart and to establish ingrowth after implantation. However, in some embodiments, the fabric may either be omitted or coupled to the base component 520 by other means than sutures that pass through apertures 524.

Still referring to FIGS. 7D-G, the base component 520 may include a main body 526 that extends upward away from the bottom surface of the base component 520. The main body 526 may follow the same general contour as the rim 522, with the rim 522 extending from the main body 526. The main body 526 may be generally circular but for a generally “U”-shaped section that defines a portion of the slot 530. In addition, the main body 526 may include two slots 540 that are substantially identical to each other but having mirrored or symmetric orientations about a centerline that bisects slot 530. In the illustrated embodiment, the two slots 540 open to the outer perimeter of the main body 526 such that the two slots 540, in addition to slot 530, interrupt the otherwise circular profile of the main body 526. As best shown in FIG. 7E, the entire lengths of the two slots 540 do not extend through the bottom surface of the base component 520, but rather are bounded at the bottom by an upper surface which is substantially flush with the upper surface of rim 522. However, as best shown in FIGS. 7E-F, an opening 541 may be positioned within each of the two slots 540, with the openings 541 opening through the bottom of the base component 520. In the illustrated embodiment, openings 541 have a generally square shape to match a shape of a post 588 of extension arms 580 so that one post 588 can be received in each opening 541, described in greater detail below. However, it should be understood that posts 588 and openings 541 may have other shapes that are complementary to each other, although preferably when a post 588 is received within an opening 541, rotation of the post 588 relative to the opening 541 is prevented.

FIGS. 7H-L are bottom perspective, top, top perspective, end, and sides views, respectively, of a single extension arm 580, although it should be understood that two identical extension arms 580 are preferably provided. In the illustrated example, extension arm 580 includes a contact arm 582 that has the shape of a portion of a circle or an arc. Preferably, contact arms 582 have a radius of curvature that is about equal to that of the rim 522 of base component 520. Preferable, the contact arm 582 includes a plurality of apertures 584 along the arc shape of the contact arm 582. The apertures 584 may be configured to receive sutures therethrough. Although not shown in FIGS. 7H-L, a fabric or other sealing member may be positioned along the bottom surface of the contact arm 582 and coupled to the contact arm 582 via suturing through the apertures 584. The fabric or sealing member may also have an arc shape similar to the contact arm 582 so that the fabric or sealing member substantially matches the shape of the contact arm 582. It should be understood that the “bottom” surface of the contact arm 582 is the surface most prominently visible in FIG. 7H, which is the surface intended to contact the patient's heart when the anchor device 500 in the expanded condition with the extension arms 580 in the extended condition, as shown in FIGS. 7A-C.

Still referring to FIGS. 7H-L, a support beam 586 may extend radially inwardly (in a direction toward the center defined by the radius of curvature of the arc-shaped contact arm 582), preferably connecting to the contact arm 582 near a center thereof. As best shown in FIG. 7H, the support beam 586 may be generally rectangular and have a width about equal to, or slightly less than, a width of the two slots 540. The support beam 586 may connect to, and terminate at, post 588. In the illustrated embodiment, post 588 may have a cross-sectional shape of a square and may be sized to fit within opening 541. However, as described above, the post 588 may have a different cross-sectional shape, preferably as long as the shape is complementary to that of openings 541. As best shown in FIGS. 7H and 7K, a bottom surface of the post 588 is preferably substantially coplanar with a bottom surface of the contact arm 582. The top surface of the post 588, on the other hand, may extend above the top surface of the contact arm 582. As best shown in FIG. 7I, post 588 preferably has a width that is about the same as the width of support beam 586, so that the support beam 586 and post 588 may both be slidably received within a corresponding one of the two slots 540.

As best shown in FIG. 7H, a recess 590 may be formed between the bottom surface of the support beam 586 and side surfaces of the post 588 and the contact arm 582. Furthermore, as bet shown in FIGS. 7H-I, one or more (two in the illustrated embodiment) detents 592 may be formed in the beam 586. In the illustrated embodiment, one detent 592 is formed in each beam surface between the top and bottom surfaces of the beam 586.

Referring back to FIGS. 7A-C, the extension arms 580 may be configured to move between two generally discrete positions. In the expanded condition of the anchor device 500, the extension arms 580 are extended so that the contact arms 582 extend beyond the outer perimeter of the rim 522 of base member 520. Notably, in this position, as best shown in FIG. 7C, the bottom surface of the extension arms 580 are substantially coplanar with the bottom surface of the base member 520. As noted above, the bottom surfaces of both the extension arms 580 and base member 520 may be provided with fabric or another sealing member so that, in the extended position, the anchor device 500 presents a relatively large surface area of contact with the heart, preferably with fabric or another sealing member disposed between the anchor device 500 and the heart to provide for enhanced short and long term sealing. When in the extension arms 580 are in the extended position, a portion of the rim 522 may be received within the groove or recess 590 of the extension arm 580, with the bottom of the post 588 received within the opening. This configuration may lead to a stable condition in which the extension arm 580 is effectively locked in the extended condition in the absence of applied forces, although the extension arms 580 may still be manually manipulated.

The second discrete position of the extension arms 580 is a contracted condition (not shown) in which the contact arms 582 are generally aligned with the perimeter of the rim 522 of base member 520. When in this contracted condition, the posts 588 are positioned radially inward compared to the position shown in FIGS. 7A-C. In this condition, the posts 588 are no longer received within openings 541, and thus the bottom surfaces of the posts 588 are in contact with a top surface of the slots 540. Due to this positioning, the bottom surfaces of the contact arms 582 are positioned upward compared to that shown in FIGS. 7A-C, so that the contact arms 582 may nest between the top of the rim 522 and the bottom of a surface of the cap 560 (described in greater detail below). In order to help maintain the extension arms 580 in the contracted condition, a biasing member may be provided. For example, as best shown in FIGS. 7C-E, the base member 520 may include two channels 528 that lead to corresponding ones of the two slots 540, for example in alignment with openings 541. A biasing member, such as a spring, may be positioned within each channel, and a ball or pin may be in contact with the spring so as to bias the ball or pin toward the slot 540. When the extension arms 580 are in the contracted position, the spring bias may push the ball or pin into one of the detents 592. When the extension arms 580 are in the extended position, the spring bias may push the ball or pin into a hole 594 in the post 588. Preferably, the force provided by the spring is enough to help maintain the extension arms 580 in the desired discrete position, but the force is not so great so that a user may manually override the spring force to manually move the extension arms 580 between the extended and contracted positions. Although a spring with a ball or pin is described above, it should be understood that various other types of biasing mechanisms may be used instead.

Referring now to FIGS. 7M-O, different views of cap 560 are shown. Cap 560 may include a ring portion 562 and a top plate 564. The ring portion 562 is not a complete circle, but rather is interrupted by a gap 566. When the cap 560 is assembled to the base member 520, as shown in FIGS. 7A-C, the gap 566 aligns with the slot 530 so that a tether may be laterally slid toward or away from the center of the anchor device 500. Further, when the cap 560 is assembled to the base member 520 and the extension arms 580, the ring portion 562 effectively creates an end wall for each of the two slots 540, which may help ensure that the extension arms 580 cannot be pulled farther radially outward relative to the base member 520 compared to the extended condition shown in FIGS. 7A-C. The cap 560 may also help prevent the extension arms 580 from being pulled upwardly away from the base member 520. The top plate 564 may be generally semi-circular, with one or more apertures 568 formed therein. In the illustrated embodiment, two apertures 568 are provided in the top plate 564, and each aperture 568 may be sized, shaped, and positioned to complement apertures 527 in the base member 520. These apertures 568, 527 may be utilized to help secure the cap 560 to the base member 520, for example via a screw, bolt, or other fastener.

Although not shown, a pin or other mechanism may be provided within the base member 520 so that, when a tether is positioned within the slot, for example at the end of the slot 530 near the radial center of the anchor device 500, the tether may be pinned or locked, preferably in a reversible manner, to couple the tether to the anchor device 500. In some embodiments, the tether holding mechanism may be any one of those disclosed in U.S. Patent Application Publication No. 2021/0298894 (“the '894 Pub”), the disclosure of which is hereby incorporated by reference herein. For example, a flap mechanism similar to that disclosed in connection with FIG. 22 of the '894 Pub may be coupled to the center area of the base member 520, for example using apertures 529.

In use, a prosthetic mitral valve PMV may be implanted as described above such that, while the prosthetic mitral valve PMV is seated within the native valve annulus, a tether coupled to the prosthetic mitral valve extends through a wall of the heart, for example at the left ventricular apex, so that the tether is accessible to the surgical personnel. Instead of needing to thread or otherwise pass an end of the tether through a closed aperture (e.g., as is the case with anchor device 200), the tether may be laterally slid through the slot 530 of the anchor device 500 until the tether is near the end of the slot, which may be near a radial center of the anchor device 500. The tether may then be tensioned and coupled using the particular pinning or connecting mechanism provided with anchor device 500. In this position, the bottom surface of the anchor device 500, which preferably includes a fabric thereon, is pressed against the wall of the heart and preferably applying pressure to the incision previously formed in the wall of the heart. Thus, the anchor device 500 interacts with the tether to maintain the tether at the desired tension, which may provide stability to the prosthetic mitral valve PMV within the native annulus, while also helping to stop any bleeding at the incision site.

In some instances, after implantation, it may be determined that the force of the tether pulling the anchor device 500 toward the heart tissue is too large for the area over which the force is acting, which may result in “dimpling” of the heart tissue. If this occurs, the anchor device 500 may be accessed in a later procedure, and the extension arms 580 may be pulled outwardly (e.g. by manually pulling the extension arms) to the extended condition shown in FIGS. 7A-C, until the bottom of the extension arms 580 also contact heart tissue, spreading the force over a greater surface area. Although the procedure is described above as being initially performed with the extension arms 580 in the contracted position, it should be understood that the extension arms may be transitioned to the extended condition prior to the initial implantation, such that the larger surface area of contact is in place after the initial implant procedure. In other words, while anchor device 500 may allow for a future intervention to switch the anchor device to the expanded condition to a greater surface area of contact, the anchor device 500 also allows for a single anchor device that may be used in the initial implantation in either the contracted or extended condition, providing an anchor device that has two different usable sizes without needing two differently sized individual anchor devices.

Although certain mechanisms are described in connection with anchor device 500 that allow for transitioning between the extended and contracted condition via application of manual pulling or pushing force, other mechanisms may be suitable. For example, a spring-loaded mechanism may be provided in which springs are compressed when the extension arms are in the contracted condition, and a button may be depressed to release the spring force to snap the extension arms into the extended condition. However, this is just one suitable alternate mechanism, and others may be used without departing from the scope of the invention.

As described above, when using an epicardial anchor device that is generally rigid and/or non-collapsible, the size of the incision required to pass the anchor device through the patient and into contact with the patient's heart is generally closely related to the size of the anchor device itself. It is generally desirable to minimize the size of the incision required to implant a prosthetic mitral valve PMV and an associated epicardial anchor device, but it is important that the effectiveness of the anchor device is not reduced as a result of a design change to reduce the size of the anchor. The epicardial anchor device 400 described in connection with FIGS. 6A-J may allow for a relatively small incision to allow the anchor device to pass into the patient and into contact with the patient's heart, without reducing the effectiveness of the anchor.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

1. An epicardial anchor device comprising:
a base defining a radial slot; and
a top component including a first piece and a second piece each operably coupled to the base, the first piece including a first recess and the second piece including a second recess, the first and second recesses facing each other in an assembled condition of the epicardial anchor device;
the epicardial anchor device having (i) an unpinned condition in which the first piece and the second piece are spaced from each other a first distance so that a tether may be laterally slid within the radial slot and between the first piece and the second piece, and (ii) a pinned condition in which the first piece and the second piece are spaced from each other a second distance so that the tether may be confined within an aperture defined by the first recess and the second recess, the second distance being smaller than the first distance.

2. The epicardial anchor device of claim 1, further comprising a pin coupled to the first piece and extending into the first recess.

3. The epicardial anchor device of claim 2, wherein in the pinned condition, the pin traverses the aperture defined by the first recess and the second recess, a free end of the pin being received within a pin channel defined by the second piece, the pin channel being adjacent to the second recess.

4. The epicardial anchor device of claim 2, further comprising a rail having a first end and a second end, wherein in the assembled condition of the epicardial anchor device, the first end of the rail is received within a first rail channel defined by the first piece, and the second end of the rail is received within a second rail channel defined by the second piece.

5. The epicardial anchor device of claim 4, wherein in the absence of applied force, engagement of the rail with the first piece and with the second piece prevents the epicardial anchor device from transitioning between the pinned condition and the unpinned condition.

6. The epicardial anchor device of claim 5, wherein the rail includes a plurality of troughs and peaks, and each of the first rail channel and the second rail channel include a plurality of troughs and peaks, the peaks of the rail being sized and shaped to be received within the troughs of the first rail channel and the troughs of the second rail channel.

7. The epicardial anchor device of claim 6, wherein the peaks of the rail are deformable so that, upon application of applied force to the first piece and/or the second piece, the peaks of the rail displace from the corresponding troughs of the first rail channel and the second rail channel to adjacent troughs of the first rail channel and the second rail channel.

8. The epicardial anchor device of claim 2, wherein the base includes a first slot on a first side of the radial slot and a second slot on a second side of the radial slot opposite the first side, the first piece having a first protrusion configured to be received within the first slot, and the second piece having a second protrusion configured to be received within the second slot.

9. An epicardial anchor device comprising:
a base component having a base with a bottom surface configured to contact a heart of a patient, the base defining an outer perimeter, the base component defining a tether-receiving passageway configured to receive a tether therethrough; and
a plurality of arms hingedly coupled to the base, the plurality of arms being rotatable relative to the base between (i) a stowed condition in which none of the plurality of arms extend radially outward beyond the outer perimeter of the base and (ii) a deployed condition in which each of the plurality of arms extend radially outward beyond the outer perimeter of the base.

10. The epicardial anchor device of claim 9, wherein each of the plurality of arms has a main body portion and a free end portion angled relative to the main body portion.

11. The epicardial anchor device of claim 10, wherein in the deployed condition of the plurality of arms, the free end portion of each of the plurality of arms is positioned to contact the heart of the patient while the bottom surface of the base of the base component simultaneously contacts the heart of the patient.

12. The epicardial anchor device of claim 9, further comprising a plurality of hinge posts extending from the base of the base component in an upward direction away from the bottom surface of the base of the base component, each of the plurality of arms being hingedly coupled to the base at corresponding ones of the hinge posts.

13. The epicardial anchor device of claim 12, wherein each of the plurality of hinge posts includes two protrusions, and each of the plurality of arms includes two loops configured to receive the two protrusions of a corresponding one of the plurality of hinge posts.

14. The epicardial anchor device of claim 9, wherein the base component includes a center post extending in an upward direction away from the bottom surface of the base of the base component, the tether-receiving passageway extending through the center post.

15. The epicardial anchor device of claim 14, further comprising a cover, the cover receiving the center post therethrough, the cover having a plurality of extensions extending radially outward from the center post, a gap being defined between each circumferentially adjacent pair of extensions.

16. The epicardial anchor device of claim 15, wherein in an unlocked condition of the cover and in the stowed condition of the plurality of arms, each arm is positioned within a corresponding gap of the cover.

17. An epicardial anchor device comprising:
a base defining (i) a radial tether slot extending through a top and bottom surface of the base and (ii) two arm slots extending through the top surface of the base;
two extension arms that each have an arcuate contact arm, a post, and a beam extending between the contact arm and the post, each post being sized and shaped to be received within and slide along a corresponding one of the two arm slots; and
a cap mounted to the base so that the two extension arms are positioned at least partially between the cap and the base,
wherein the anchor device has (i) a contracted position in which the posts of the extension arms are relatively close to a longitudinal center of the base and the contact arms are aligned with an outer perimeter of the base, and (ii) an extended position in which the posts of the extension arms are relatively far from the longitudinal center of the bae and the contact arms extend beyond the outer perimeter of the base.

18. The epicardial anchor device of claim 17, wherein in the extended position, a bottom surface of each of the extension arms is substantially coplanar with the bottom surface of the base.

19. The epicardial anchor device of claim 18, wherein the beam of each of the two extension arms has at least one groove, and a biasing mechanism being positioned within the base, the biasing mechanism interacting with the at least one groove to maintain the anchor device in the contracted position in the absence of applied forces.

20. The epicardial anchor device of claim 18, wherein each of the two arm slots includes an opening that opens to the bottom surface of the base, the post of each of the two extension arms being received within a corresponding one of the openings when the anchor device is in the extended position.
